(19) United States
(12) Patent Application Publication
(10) Pub. No.: US 2017/0305832 A1
(43) Pub. Date: Oct. 26, 2017

(54) PREPARATION OF A SORBATE ESTER

(71) Applicants: Dow Global Technologies LLC, Midland, MI (US); Rohm and Haas Company, Philadelphi, PA (US)

(72) Inventors: Selvanathan Arumugam, Blue Bell, PA (US); John Ell, Quakertown, PA (US); Bo Lu, Shanghai (CN); Brandon Rowe, Robbinsville, NJ (US); Jiguang Zhang, Shanghai (CN)

(21) Appl. No.: 15/521,353

(22) PCT Filed: Oct. 22, 2014
(86) PCT No.: PCT/CN2014/089153

§ 371 Date: Apr. 24, 2017

(51) Int. Cl. C07C 67/26; C07C 69/587; C07C 67/58

(52) U.S. Cl. CPC C07C 67/26; C07C 67/58; C07C 69/587

(57) ABSTRACT

The present invention is a process for preparing a sorbate ester comprising the steps of: a) contacting together in a reaction vessel a first organic solvent, sorbic acid, a catalytic amount of TiC14, an anti-oxidant, and an alkylene oxide under conditions sufficient to form a mixture of the sorbate ester and residual water-soluble impurities; then b) separating the sorbate ester from the residual water-soluble impurities by washing. Sorbate esters can be prepared in an efficient and cost-effective manner by the process of the present invention.

### BACKGROUND OF THE INVENTION

The present invention relates to the preparation of a sorbate ester, which is useful as a reactive coalescent in coatings formulations.

Sorbic esters have recently been shown to be suitable as reactive coalescents that promote significant improvement in the coating hardness and tack in waterborne architectural coating formulations. A sorbic ester of particular interest is sorbic PO, which can be prepared by the FeCl₃ catalyzed reaction of sorbic acid and propylene oxide, as disclosed by Masahiro et al. in EP0387654A2. Masahiro teaches that direct purification of the product by distillation is problematic because “the heat transfer surface of a distillation apparatus is contaminated by catalyst and the long term operation becomes impossible.” Consequently, multiple washing steps are required prior to distillation. Accordingly, it would be an advance in the art to find a more efficient and cost effective way of preparing hydroxypropyl sorbate as well as other sorbate esters, particularly high molecular weight non-volatile sorbate esters.

### SUMMARY OF THE INVENTION

The present invention addresses a need in the art by providing a process for preparing a sorbate ester comprising the steps of: a) contacting together in a reaction vessel a first organic solvent, sorbic acid, a catalytic amount of a titanate catalyst, an anti-oxidant, and an alkylene oxide selected from the group consisting of C₂-C₄ alkylene oxides, glycidyl methacrylates, glycidyl acrylates, diglycidyl ethers of bisphenols, alkylene glycol diglycidyl ethers, polyalkylene glycol diglycidyl ethers, alkane diol diglycidyl ethers, tri- and tetraglycidyl ethers, and hydoxylalkyl alkylene oxides under conditions sufficient to form a mixture of the sorbate ester and residual water-soluble impurities; then b) separating the sorbate ester from the residual water-soluble impurities by washing; wherein the anti-oxidant is a hindered amine, a hindered N-oxide, a phenothiazine, or a phenol, or a combination thereof.

Sorbate esters can be prepared in an efficient and cost-effective manner by the process of the present invention.

### DETAILED DESCRIPTION OF THE INVENTION

The present invention is a process for preparing a sorbate ester comprising the steps of: a) contacting together in a reaction vessel a first organic solvent, sorbic acid, a catalytic amount of a titanate catalyst, an anti-oxidant, and an alkylene oxide selected from the group consisting of C₂-C₄ alkylene oxides, glycidyl methacrylates, glycidyl acrylates, diglycidyl ethers of bisphenols, alkylene glycol diglycidyl ethers, polyalkylene glycol diglycidyl ethers, alkane diol diglycidyl ethers, tri- and tetraglycidyl ethers, and hydoxylalkyl alkylene oxides under conditions sufficient to form a mixture of the sorbate ester and residual water-soluble impurities; then b) separating the sorbate ester from the residual water-soluble impurities by washing; wherein the anti-oxidant is a hindered amine, a hindered N-oxide, a phenothiazine, or a phenol, or a combination thereof.

The C₂-C₄ alkylene oxides are ethylene oxide, propylene oxide, and butylene oxide; an example of a diglycidyl ether of a bisphenol is bisphenol A diglycidyl ether; alkylene glycols- and polyalkylene glycols of diglycidyl ethers include ethylene- and polyethylene glycol diglycidyl ethers, propylene- and polypropylene glycol diglycidyl ethers, and butylene- and polybutylene glycol diglycidyl ethers; alkane diol diglycidyl ethers include 1,4-butane diol diglycidyl ether and 1,6-hexane diol diglycidyl ether; tri- and tetraglycidyl ethers include glycerine triglycidyl ether, pentaerythrite tetraglycidyl ether, and trimethylol propane triglycidyl ether; and hydroxylalkyl alkylene oxides include glycidol and glycerol diglycidyl ether. A preferred alkylene oxide is propylene oxide. As used herein, a hydroxypropyl sorbate is either 2-hydroxypropyl sorbate or 2-hydroxy-l-methylethyl sorbate, or a combination thereof.

The organic solvent is preferably a nonpolar solvent, examples of which include butyl acetate, xylenes, toluene, and mesitylene. The catalyst is used in a sufficient amount to promote the conversion of the sorbic acid and the alkylene oxide to the hydroxypropyl sorbate, preferably from 0.1, more preferably from 0.5 weight percent, to preferably 5, more preferably to 3 weight percent, based on the weight of the sorbic acid and the alkylene oxide.

As used herein, a titanate catalyst is a halogenated or an alkoxylated titanium catalyst, examples of which include TiCl₄, TiBr₄, Ti(O-n-butyl)₄, and Ti(O-isopropyl)₄, with TiCl₄ being preferred. The anti-oxidant is preferably used at a concentration of from 10 ppm, more preferably from 20 ppm, and most preferably from 50 ppm, to 1 weight percent, more preferably to 0.5 weight percent, and most preferably to 0.1 weight percent, based on the weight of the sorbic acid and the propylene oxide. The radical mediator may be a hindered amine, a hindered N-oxide, or a phenol, or a combination thereof. As used herein, a hindered amine is a protonated secondary amine attached to two tertiary or quaternary saturated carbon atoms, as illustrated:

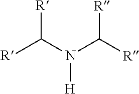

wherein R′ and R″ are each independently H or a C₁-C₁₂-alkyl group with the proviso that at least one of the R′ groups and one of the R″ groups is a C₁-C₁₂-alkyl group optionally functionalized with a hydroxyl group or an ether group, or one of the R′ groups and one of the R″ groups together with the carbon atoms to which they are attached form a piperidine ring or a pyrrolidine ring, either unsubstituted or substituted with a hydroxyl group or an ether group. Examples of suitable hindered amine radical mediators include 2,6-dimethyl piperidine and 2,2,6,6-tetramethyl piperidine.

Similarly, a hindered N-oxide can be characterized by the following formula:

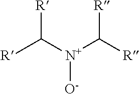

Examples of suitable hindered N-oxides include 2,2,6,6-tetramethylpiperidine-1-oxyl (TEMPO), 4-acetamido-2,2,6,6-tetramethylpiperidine-1-oxyl (4-acetamido TEMPO), and 4-hydroxy-2,2,6,6-tetramethylpiperidine-1-oxyl (4-hydroxy TEMPO). Examples of suitable phenols include phenol, hydroxytoluenes, and p-methoxyphenol (also known as hydroquinone monomethyl ether or MEHQ).

The first organic solvent, sorbic acid, TiC14, and anti-oxidant are advantageously contacted together in a reaction vessel at an advanced temperature, preferably in a range of from 50° C., more preferably from 60° C., to preferably 140° C., more preferably to 90° C., prior to introduction of the alkylene oxide to the reaction vessel. The alkylene oxide, preferably propylene oxide, is preferably added slowly to a mixture of the first organic solvent, sorbic acid, TiCl₄, and anti-oxidant to prevent the formation of oligomeric byproducts and to control the reaction exotherm. The alkylene oxide is preferably added in a stoichiometric excess so that the sorbic acid is the limiting reagent.

The reaction is preferably carried out to substantial completion to form a mixture of the desired product and residual water-soluble impurities. As used herein, water-soluble impurities are undesirable materials that are extractable by water, preferably water at a pH of greater than 7, more preferably greater than 8.

Upon substantial completion, the reaction is advantageously quenched with water, followed by filtration to filter out any undissolved particles. Then, the water-soluble impurities are separated from the hydroxypropyl sorbate by washing. In a preferred washing method, the product mixture is contacted with a suitable second organic solvent (which may be the same as or different from the first organic solvent) and aqueous base to form a bilayer of an aqueous phase and an organic phase. The organic phase is preferably washed multiple times with aqueous base to remove the residual water-soluble impurities. Optionally, additional product may be obtained by extraction of the water fractions with the second organic solvent. The organic phase or phases are then advantageously dried over a suitable drying agent such as Na₂SO₄, followed by filtration and solvent removal in vacuo as a preferred final step.

A second anti-oxidant is advantageously added to the purified product after purification to achieve storage stability. Any suitable anti-oxidant or combinations of anti-oxidants would be effective for this purpose; for example, from 10 ppm to 5000 ppm of hindered N-oxides, preferably TEMPO ((2,2,6,6-tetramethylpiperidin-l-yl)oxidanyl) or 4-hydroxy TEMPO, more preferably 4-hydroxy TEMPO, or hindered phenols such as 2,6-bis(1,1-dimethylethyl)-4-methylphenol are added to the product after purification. More preferably, the addition of a combination of hindered N-oxides and hindered phenols are found to be particularly effective for providing long term storage stability.

The process of the present invention provides a way to produce the described sorbate esters at high yield (˜98%) and high purity without the need for a final product distillation step.

### EXAMPLE

Preparation of Hydroxypropyl Sorbate

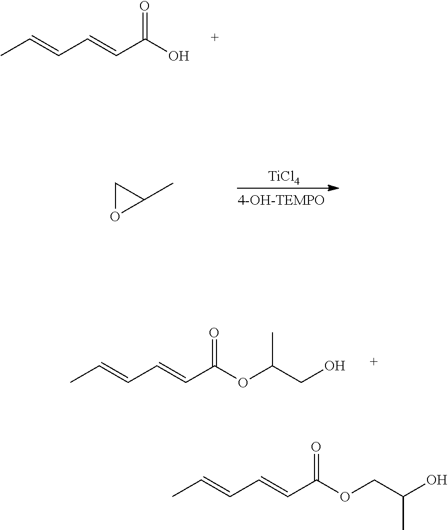

To a three-neck flask purged with N2 was added sorbic acid (50 g, 0.45 mol), 4-hydroxyTEMPO (50 mg), toluene (100 mL), and TiCl₄ (2.47 mL, 4.27 g, 0.0225 mol). The resulting mixture was heated to 75° C., and propylene oxide (61.4 mL, 51 g, 0.88 mol) was added dropwise. After 6 h, the reaction was quenched with water (5 mL) and white precipitate (presumably hydrolyzed TiCl₄) was observed and filtrated with celite. The flask and precipitate were rinsed with ethyl acetate (100 mL) and the organic phase was washed three times with saturated NaHCO₃ aq (100 mL×3) to remove residual water-soluble impurities. The combined aqueous phases were extracted with ethyl acetate (200 mL) and the organic phases were combined and washed with brine (100 mL) then dried over Na₂SO₄. Filtration and concentration afforded the final product hydroxypropyl sorbate (75 g, 98% yield) as a light yellow, low viscosity liquid.

1. A process for preparing a sorbate ester comprising the steps of: a) contacting together in a reaction vessel a first organic solvent, sorbic acid, a catalytic amount of a titanate catalyst, an anti-oxidant, and an alkylene oxide selected from the group consisting of C₂-C₄ alkylene oxides, glycidyl methacrylates, glycidyl acrylates, diglycidyl ethers of bisphenols, alkylene glycol diglycidyl ethers, polyalkylene glycol diglycidyl ethers, alkane diol diglycidyl ethers, tri- and tetraglycidyl ethers, and hydoxylalkyl alkylene oxides under conditions sufficient to form a mixture of the sorbate ester and residual water-soluble impurities; then b) separating the sorbate ester from the residual water-soluble impurities by washing; wherein the anti-oxidant is a hindered amine, a hindered N-oxide, a phenothiazine, or a phenol, or a combination thereof.

2. The process of claim 1 wherein the solvent, sorbic acid, and the titanate catalyst are contacted together prior to the introduction of the alkylene oxide.

3. The process of claim 1 wherein the alkylene oxide is ethylene oxide, propylene oxide, butylene oxide, bisphenol A diglycidyl ether, ethylene glycol diglycidyl ether, polyethylene glycol diglycidyl ethers, propylene glycol diglycidyl ether, polypropylene glycol diglycidyl ethers, butylene glycol diglycidyl ether, polybutlene glycol diglycidyl ethers, 1,4-butane diol diglycidyl ether, 1,6-hexane diol diglycidyl ether, glycerine triglycidyl ether, pentaerythrite tetraglycidyl ether, trimethylol propane triglycidyl ether, glycidol, or glycerol diglycidyl ether.

4. The process of claim 1 wherein the alkylene oxide is propylene oxide.

5. The process of claim 4 wherein the anti-oxidant is TEMPO or 4-hydroxy TEMPO and the titanate catalyst is TiCl₄.

6. The process of claim 5 which is carried out in toluene at a temperature in the range of 60° C. to 90° C.

7. The process of claim 1 wherein between steps a) and b), the mixture of the sorbate ester and residual water-insoluble impurities is quenched with water, followed by filtration to remove any insoluble impurities.

8. The process of claim 1 wherein the washing is carried out by the steps of contacting the mixture of the sorbate ester and residual water-insoluble impurities with aqueous base and a second organic solvent to form a bilayer of an organic phase and an aqueous phase, then separating the organic phase from the aqueous phase.

9. The process of claim 8 which further comprises removing organic solvents in vacuo from the organic phase as a final purification and isolation step for the sorbate ester.

10. The process of claim 9 which further includes the step of adding from 10 ppm to 5000 ppm of 4-hydroxy TEMPO or 2,6-bis(1,1-dimethylethyl)-4-methylphenol or a combination thereof to the purified sorbate ester.
